(19) United States
(12) Patent Application Publication
(10) Pub. No.: US 2007/0184433 A1
(43) Pub. Date: Aug. 9, 2007

(54) Microparticle based biochip systems and uses thereof

(76) Inventors: Shengce Tao, Beijing (CN); Guoqing Wang, Beijing (CN); Jing Cheng, Beijing (CN)

Correspondence Address: MORRISON & FOERSTER LLP, 12531 HIGH BLUFF DRIVE, SUITE 100, SAN DIEGO, CA, 92130-2040, US

(21) Appl. No.: 10/557,328

(22) PCT Filed: Jul. 14, 2003
(86) PCT No.: PCT/CN03/00562

§ 371 Date: Mar. 29, 2007

(30) Foreign Application Priority Data
May 19, 2003 (CN) 0323726.6

(51) Int. Cl. C12Q 1/70; C12Q 1/68; C12M 3/00; C07H 21/04

(52) U.S. Cl. 435/5; 435/6; 435/287.2; 536/25.32; 977/924

(57) ABSTRACT

This invention relates generally to the field of analyte assays. In particular, the invention provides a device for analyzing an analyte, which device comprises, inter alia, various means for moving analytes and other items to facilitate binding between analytes and their binding reagents immobilized on a surface and to facilitate clearance of undesirable items away from analyte-binding reagent interaction area to reduce background noise in the assay. Methods for analyzing an analyte using the devices are also disclosed.

### TECHNICAL FIELD

This invention relates generally to the field of analyte assays. In particular, the invention provides a device for analyzing an analyte, which device comprises, inter alia, various means for moving analytes and other items to facilitate binding between analytes and their binding reagents immobilized on a surface and to facilitate clearance of undesirable items away from analyte-binding reagent interaction area to reduce background noise in the assay. Methods for analyzing an analyte using the devices are also disclosed.

### BACKGROUND ART

Different methods are currently used for detecting different types of biological analytes. For example, nucleic acid hybridization, polymerase chain reaction (PCR), restriction enzyme analysis, and gel electrophoresis are conventionally used for detecting nucleic acid. For detecting proteins, immunological method and gel electrophoresis are generally used. As a revolutionary analytical method and technology, biochip is becoming more and more important in analytical biotechnologies because of its potential for integration, miniaturization, and automation. At an early stage, biochip is mainly used as nucleic acid chip or DNA microarray, which has been well developed and widely used in analytical biotechnologies. Nucleic acid chip or array can be used to assay large number of nucleic acids simultaneously (Debouck and Goodfellow, Nature Genetics, 21 (Suppl.):48-50 (1999); Duggan et al., Nature Genetics, 21 (Suppl.):10-14 (1999); Gerhold et al., Trends Biochem. Sci., 24:168-173 (1999); and Alizadeh et al., Nature, 403:503-511 (2000)). Gene expression pattern under a given condition can be rapidly analyzed using nucleic acid chip or array. The SNPs in a particular region, up to a 1 kb, can be analyzed in one experiment using nucleic acid chip or array (Guo et al., Genome Res., 12:447-57 (2002)).

A variety of different types of biochips has been developed based on the combination of the biochip concept and the basic principle of biology and conventional biological detection technology. These biochips include protein chips used for disease and cancer research (Belov et al., Cancer Research, 61:4483-4489 (2001); Knezevic et al., Proteomics, 1:1271-1278 (2001); Paweletz et al., Oncogene, 20:1981-1989 (2001)); tissue microarrays developed for genome-scale molecular pathology studies (Kononen et al., Nat. Med, 4: 844-847 (2001)); and polysaccharide microarrays for studying interactions between polysaccharides and proteins (Fukui et al., Nat. Biotech., 20: 1011-1017 (2002)).

Conventional nucleic acid based detection methods (which use passive chip), such as the method used in clinical detection for infectious agents, include three separate steps. The first step is sample preparation, e.g., treating samples, such as serum, whole blood, saliva, urine and feces, to obtain nucleic acids, e.g., DNA or RNA. Often, insufficient amount of the nucleic acids are isolated or prepared from the samples and the prepared nucleic acids are amplified using a number of methods such as polymerase chain reaction (PCR), reverse transcription polymerase chain reaction (RT-PCR), strand displacement amplification (SDA) and rolling cycle amplification (RCA), etc. (Andras et al., Mol. Biotechnol., 19:29-44 (2001)). The second step is hybridization, i.e., hybridization between amplified nucleic acid samples and probes immobilized on the biochip. The third step is to detect the hybridization signal, which is often based on the detection of a label. The label can be introduced during the amplification or hybridization step. The signal detection methods vary according to the label used, e.g., a fluorescent detector is used to detect a fluorescent label, autoradiography is used to detect a radioactive label, and detection of a biolabel, e.g., biotin label, digoxigenin label, etc., may require further enzymatic amplifications. Depending on the required detection sensitivity, various signal amplification methods can be used, e.g., Tyramide signal amplification (TSA) (Karsten et. al., Nucleic Acids Res., E4. ((2002)) and Dendrimer (Kricka Clin. Chem., 45:453-8 (1999)).

The separation of the three key steps in nucleic acid detection requires manual manipulations among these steps. Theses manual manipulations make the detection procedure complex, time consuming, costly, and may introduce experimental error, and decrease repeatability and consistency of the detection. The manual manipulations also increase cross contamination, which is a major reason that hampers wide application of nucleic acid based detection, especially any such detection comprising an amplification step, in clinical use. In addition, since the interaction between an analyte in a liquid sample and probes immobilized on a solid substrate depends on passive diffusion of the analyte in the liquid sample and the concentration of the analyte near the probes is low, the reaction efficiency is relatively low and the time required for the reaction is relatively long.

To address the problems, such as low efficiency of passive reaction and low quantity of immobilized probes, associated with the conventional 2-D (dimensional) biochip, several types of solutions have been developed. One solution is to use other types of substrates and immobilizing strategies. In gene chip technology, the probes are usually immobilized to a 2-D (dimensional) plate surface. Thus, the surface density of the probes is low. In order to achieve higher hybridization efficiency, the possibility of attaching probes to 3-D (dimensional) structures or substrates have been attempted. (Zlatanova et al., Methods Mol. Biol. 170: 17-38 (2001); Tillib et al., Anal. Biochem. 292: 155-160 (2001); Michael et al., Anal. Chem. 70:1242-1248 (1998)). Compared with traditional 2-D biochip, the 3-D chip has two new features: larger amounts of probes within a definitive spot area; higher flexibility of the probes within the structure, consequently, this type of chip can increase the hybridization efficiency. However the disadvantages are also obvious, for instance, the fabrication procedure is complex. As a consequence, this type of gene chips is difficult to be high density. Another solution is to use specially designed probes. These probes have special structures or 5′ attachments, such as 5′ spacers for improving flexibility (Shchepinov et al., Nucleic Acids Res. 25, 1155-1161 (1997)), and stem-loop probes or hairpin-structure probes (Broude et al., Nucleic Acids Res. 29: E92 (2001)). Hybridization of DNA targets to such arrays is enhanced by contiguous stacking interactions (Riccelli et al., Nucleic Acids Res. 29: 996-1004 (2001)). Another more powerful way for improving hybridization efficiency is the application of physical forces to gene chip. Agitation has been used to increase the diffusion of target during hybridization, such as the Lucidea Automated Slide Processor (Lucidea ASP). Electric force has been applied to enable rapid movement and concentration of nucleic acids on gene chip (Sosnowski et al., Proc. Natl. Acad. Sci. U.S. A 94: 1119-1123 (1997); Cheng et al., Nat. Biotechnol. 16: 541-546 (1998)). This type of chip is regarded as active chip. The later feature can accelerate molecular binding on the microchip up to 1,000 times that of the traditional passive methods. The disadvantage of this technique is the complexity of the chip and the device.

Lab-on-chip systems have been proposed to address the drawbacks of separation of reactions by the conventional chips (Manz et al., Anal. Chem. 74: 2623-2636 and 2637-2652 (2002)). Biochemical reactions and analyses often include three steps: sample preparation, biochemical reactions and signal detection and data analyses. Miniaturizing one or more steps on a chip leads to a specialized biochip, e.g., cell filtration chip and dielectrophoresis chip for sample preparation, DNA microarray for detecting genetic mutations and gene expression and high-throughput micro-reaction chip for drug screening, etc. Efforts have been made to perform all steps of biochemical analysis on chips to produce micro-analysis systems or lab-on-chip systems. Using such micro-analysis systems or lab-on-chip systems, it will be possible to complete all analytic steps from sample preparation to obtain analytical results in a closed system rapidly.

One drawback of the current lab-on-chip systems is its requirement of complex micro-scale engineering, which is technologically demanding. Most of the reported lab-on-chip systems are based on the miniaturization of a particular step, e.g., sample preparation chip, (Wilding et al., Anal. Biochem., 257:95-100 (1998), cell isolation chip (Wang et al., J. Phys. D: Appl. Phys., 26:1278-1285 (1993) and PCR chip (Cheng et al., Nucleic Acids Res., 24:380-385 1996). Cheng et al. reported a first lab-on-chip system that integrates the sample preparation, biochemical reaction and result detection together (Cheng et al., Nat. Biotechnol., 16:541-546 (1998)), which has not been commercialized. The currently commercialized system, e.g., Nanogen's Microelectronic Array, only integrates and automates the hybridization and signal detection steps. A set of complex instruments and analytical software must be used with the Nanogen's Microelectronic Array. In addition, the cost for making and using Nanogen's electrophoresis chip is high.

The present invention addresses the above and other related concerns in the art.

### DISCLOSURE OF THE INVENTION

In one aspect, the present invention is directed to a device for analyzing an analyte, which device comprises: a) a controllably closed space enclosed by a suitable material on a substrate, wherein said suitable material is thermoconductive, biocompatible and does not inhibit binding between an analyte and a reactant, and said controllably closed space comprising, on the surface of said substrate, a first immobilized reactant capable of binding to said analyte; b) a first means for controllably moving said analyte to said first immobilized reactant; c) a second means for controllably moving said analyte to a labeled unimmobilized complex comprising a second reactant capable of binding to said analyte and a microparticle; and d) a third means for controllably moving said labeled unimmobilized complex unbound to said analyte away from said first immobilized reactant, and wherein addition of a sample comprising said analyte and said labeled unimmobilized complex into said controllably closed space and operation of said means result in formation of a sandwich of said labeled unimmobilized complex-said analyte-said first immobilized reactant on said substrate, preferably without any material exchange between said controllably closed space and the outside environment.

In another aspect, the present invention is directed to a method for analyzing an analyte, which method comprises: a) providing an above-described device; b) introducing a sample containing or suspected of containing an analyte and a labeled unimmobilized complex comprising a second reactant capable of binding to said analyte and a microparticle into said controllably closed space of said device; c) operating said means of said device to form a sandwich of said labeled unimmobilized complex-said analyte-said first immobilized reactant on said substrate of said device, preferably without any material exchange between said controllably closed space and the outside environment; d) assessing said sandwich to determine presence and/or quantity of said analyte in said sample.

### BRIEF DESCRIPTION OF THE DRAWINGS

FIG. 1 illustrates an exemplary device of the present invention.

FIGS. 2-1 to 2-9 illustrate an exemplary operation of the device illustrated in FIG. 1 to bind various analytes to their binding reagents on the surface of the device and to remove undesirable items from the analyte-binding reagent interaction area.

FIG. 3 illustrates an exemplary operation of the device illustrated in FIG. 1 wherein the microparticle are manipulated via centrifugational or magnetic forces. In one example, the microparticle are polystyrene microparticle. These microparticle are concentrated to the reactant, e.g., probe, areas via centrifugational force. when the side of the substrate having the reactants faces up, the area having the reactants sags relative to other part of the substrate as shown in FIG. 3, which facilitate enrichment of the microparticle around the reactants under centrifugational force.

### MODES OF CARRYING OUT THE INVENTION

For clarity of disclosure, and not by way of limitation, the detailed description of the invention is divided into the subsections that follow.

### A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “a second means for controllably moving said analyte to a labeled unimmobilized complex” means that the means can move the analyte to the labeled unimmobilized complex or move the labeled unimmobilized complex to the analyte or move both the analyte and the labeled unimmobilized complex to a third item or location.

As used herein, “specific binding” refers to the binding of one material to another in a manner dependent upon the presence of a particular molecular structure. For example, a receptor will selectively bind ligands that contain the chemical structures complementary to the ligand binding site(s).

As used herein, “specific binding pair” refers to any substance, or class of substances, which has a specific binding affinity for the ligand to the exclusion of other substances. In one embodiment, the specific binding pair includes specific binding assay reagents which interact with the sample ligand or the binding capacity of the sample for the ligand in an immunochemical manner. For example, there will be an antigen-antibody or hapten-antibody relationship between reagents and/or the sample ligand or the binding capacity of the sample for the ligand. Additionally, it is well understood in the art that other binding interactions between the ligand and the binding partner serve as the basis of specific binding assays, including the binding interactions between hormones, vitamins, metabolites, and pharmacological agents, and their respective receptors and binding substances. (See e.g., Langan et al. eds., Ligand Assay, pp. 211 et seq., Masson Publishing U.S.A. Inc., New York, 1981).

As used herein, “antibody” refers to specific types of immunoglobulin, i.e., IgA, IgD, IgE, IgG, e.g., IgG₁, IgG₂, IgG₃, and IgG₄, and IgM. An antibody can exist in any suitable form and also encompass any suitable fragments or derivatives. Exemplary antibodies include a polyclonal antibody, a monoclonal antibody, a Fab fragment, a Fab′ fragment, a F(ab′)₂ fragment, a Fv fragment, a diabody, a single-chain antibody and a multi-specific antibody formed from antibody fragments.

As used herein, “plant” refers to any of various photosynthetic, eucaryotic multi-cellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth and fixed bodily structure. Non-limiting examples of animals include birds such as chickens, vertebrates such fish and mammals such as mice, rats, rabbits, cats, dogs, pigs, cows, ox, sheep, goats, horses, monkeys and other non-human primates.

As used herein, “bacteria” refers to small prokaryotic organisms (linear dimensions of around 1 micron) with non-compartmentalized circular DNA and ribosomes of about 70S. Bacteria protein synthesis differs from that of eukaryotes. Many anti-bacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are three main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “fungus” refers to a division of eucaryotic organisms that grow in irregular masses, without roots, stems, or leaves, and are devoid of chlorophyll or other pigments capable of photosynthesis. Each organism (thallus) is unicellular to filamentous, and possesses branched somatic structures (hyphae) surrounded by cell walls containing glucan or chitin or both, and containing true nuclei.

As used herein, “virus” refers to an obligate intracellular parasite of living but non-cellular nature, consisting of DNA or RNA and a protein coat. Viruses range in diameter from about 20 to about 300 mm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause in plants, animals and prokaryotes. Viruses of prokaryotes are known as bacteriophages.

As used herein, “tissue” refers to a collection of similar cells and the intracellular substances surrounding them. There are four basic tissues in the body: 1) epithelium; 2) connective tissues, including blood, bone, and cartilage; 3) muscle tissue; and 4) nerve tissue.

As used herein, “organ” refers to any part of the body exercising a specific function, as of respiration, secretion or digestion.

As used herein, “sample” refers to anything which may contain an analyte to be analyzed using the present devices and/or methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “magnetic substance” refers to any substance that has the properties of a magnet, pertaining to a magnet or to magnetism, producing, caused by, or operating by means of, magnetism.

As used herein, “magnetizable substance” refers to any substance that has the property of being interacted with the field of a magnet, and hence, when suspended or placed freely in a magnetic field, of inducing magnetization and producing a magnetic moment. Examples of magnetizable substances include, but are not limited to, paramagnetic, ferromagnetic and ferrimagnetic substances.

As used herein, “paramagnetic substance” refers to the substances where the individual atoms, ions or molecules possess a permanent magnetic dipole moment. In the absence of an external magnetic field, the atomic dipoles point in random directions and there is no resultant magnetization of the substances as a whole in any direction. This random orientation is the result of thermal agitation within the substance. When an external magnetic field is applied, the atomic dipoles tend to orient themselves parallel to the field, since this is the state of lower energy than antiparallel position. This gives a net magnetization parallel to the field and a positive contribution to the susceptibility. Further details on “paramagnetic substance” or “paramagnetism” can be found in various literatures, e.g., at Page 169-page 171, Chapter 6, in “Electricity and Magnetism” by B. I Bleaney and B. Bleaney, Oxford, 1975.

As used herein, “ferromagnetic substance” refers to the substances that are distinguished by very large (positive) values of susceptibility, and are dependent on the applied magnetic field strength. In addition, ferromagnetic substances may possess a magnetic moment even in the absence of the applied magnetic field, and the retention of magnetization in zero field is known as “remanence”. Further details on “ferromagnetic substance” or “ferromagnetism” can be found in various literatures, e.g., at Page 171-page 174, Chapter 6, in “Electricity and Magnetism” by B. I Bleaney and B. Bleaney, Oxford, 1975.

As used herein, “ferrimagnetic substance” refers to the substances that show spontaneous magnetization, remanence, and other properties similar to ordinary ferromagnetic materials, but the spontaneous moment does not correspond to the value expected for full parallel alignment of the (magnetic) dipoles in the substance. Further details on “ferrimagnetic substance” or “ferrimagnetism” can be found in various literatures, e.g., at Page 519-524, Chapter 16, in “Electricity and Magnetism” by B. I Bleaney and B. Bleaney, Oxford, 1975.

As used herein, “metal oxide particle” refers to any oxide of a metal in a particle form. Certain metal oxide particles have paramagnetic or super-paramagnetic properties. “Paramagnetic particle” is defined as a particle which is susceptible to the application of external magnetic fields, yet is unable to maintain a permanent magnetic domain. In other words, “paramagnetic particle” may also be defined as a particle that is made from or made of “paramagnetic substances”. Non-limiting examples of paramagnetic particles include certain metal oxide particles, e.g., Fe₃O₄ particles, metal alloy particles, e.g., CoTaZr particles.

As used herein: “stringency of hybridization” in determining percentage mismatch is as follows:

1) high stringency: 0.1×SSPE, 0.1% SDS, 65° C.;

2) medium stringency: 0.2×SSPE, 0.1% SDS, 50° C. (also referred to as moderate stringency); and

3) low stringency: 1.0×SSPE, 0.1% SDS, 50° C.

It is understood that equivalent stringencies may be achieved using alternative buffers, salts and temperatures.

As used herein, “chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, electrode elements, electromagnetic elements, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips used in the present invention can vary considerably, e.g., from about 1 mm² to about 0.25 m². Preferably, the size of the chips is from about 4 mm² to about 25 cm² with a characteristic dimension from about 1 mm to about 7.5 cm. The chip surfaces may be flat, or not flat. The chips with non-flat surfaces may include channels or wells fabricated on the surfaces. One example of a chip is a solid substrate onto which multiple types of DNA molecules or protein molecules or cells are immobilized.

As used herein the term “assessing” is intended to include quantitative and/or qualitative determination of an analyte present in the sample, and also of obtaining an index, ratio, percentage, visual or other value indicative of the level of the analyte in the sample. Assessment may be direct or indirect and the chemical species actually detected need not of course be the analyte itself but may for example be a derivative thereof or some further substance.

As used herein, “small molecule” refers to a molecule that, without forming homo-aggregates or without attaching to a macromolecule or adjuvant, is incapable of generating an antibody that specifically binds to the small molecule. Preferably, the small molecule has a molecular weight that is about or less than 10,000 daltons. More preferably, the small molecule has a molecular weight that is about or less than 5,000 dalton.

As used herein, “a controllably closed space” means that the opening and closing of the space can be controlled at will, e.g., open to the outside to allow addition of sample or other reagents and close to allow the formation of a sandwich of the labeled unimmobilized complex-the analyte-the first immobilized reactant on the substrate in the controllably closed space without any material exchange between the controllably closed space and the outside environment.

As used herein, “biocompatibility” refers to the quality and ability of a material of not having toxic or injurious effects on biological systems and biological or biochemical reactions.

As used herein, “thermal conductivity” refers to the effectiveness of a material as a thermal insulator, which can be expressed in terms of its thermal conductivity. The energy transfer rate through a body is proportional to the temperature gradient across the body and its cross sectional area. In the limit of infinitesimal thickness and temperature difference, the fundamental law of heat conduction is:
Q=λAdT/dx

wherein Q is the heat flow, A is the cross-sectional area, dT/dx is the temperature/thickness gradient and λ is defined as the thermal conductivity value. A substance with a large thermal conductivity value is a good conductor of heat, one with a small thermal conductivity value is a poor heat conductor, i.e., a good insulator. Hence, knowledge of the thermal conductivity value (units W/m·K) allows comparisons, quantitative comparisons if desirable, to be made between the thermal insulation efficiencies of different materials.

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “probe” refers to an oligonucleotide or a nucleic acid that hybridizes to a target sequence, typically to facilitate its detection. The term “target sequence” refers to a nucleic acid sequence to which the probe specifically binds. Unlike a primer that is used to prime the target nucleic acid in amplification process, a probe need not be extended to amplify target sequence using a polymerase enzyme.

As used herein, “complementary or matched” means that two nucleic acid sequences have at least 50% sequence identity. Preferably, the two nucleic acid sequences have at least 60%, 70,%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein, “melting temperature” (“Tm”) refers to the midpoint of the temperature range over which nucleic acid duplex, i.e., DNA:DNA, DNA:RNA, RNA:RNA, PNA:DNA, LNA:RNA and LNA:DNA, etc., is denatured.

As used herein, “label” refers to any chemical group or moiety having a detectable physical property or any compound capable of causing a chemical group or moiety to exhibit a detectable physical property, such as an enzyme that catalyzes conversion of a substrate into a detectable product. The term “label” also encompasses compound that inhibit the expression of a particular physical property. The “label” may also be a compound that is a member of a binding pair, the other member of which bears a detectable physical property. Exemplary labels include mass groups, metals, fluorescent groups, luminescent groups, chemiluminescent groups, optical groups, charge groups, polar groups, colors, haptens, protein binding ligands, nucleotide sequences, radioactive groups, enzymes, particulate particles, a fluorescence resonance energy transfer (FRET) label, a molecular beacon and a combination thereof.

### B. Devices for Analyzing an Analyte

In one aspect, the present invention is directed to a device for analyzing an analyte, which device comprises: a) a controllably closed space enclosed by a suitable material on a substrate, wherein said suitable material is thermoconductive, biocompatible and does not inhibit binding between an analyte and a reactant, and said controllably closed space comprising, on the surface of said substrate, a first immobilized reactant capable of binding to said analyte; b) a first means for controllably moving said analyte to said first immobilized reactant; c) a second means for controllably moving said analyte to a labeled unimmobilized complex comprising a second reactant capable of binding to said analyte and a microparticle; and d) a third means for controllably moving said labeled unimmobilized complex unbound to said analyte away from said first immobilized reactant, and wherein addition of a sample comprising said analyte and said labeled unimmobilized complex into said controllably closed space and operation of said means result in formation of a sandwich of said labeled unimmobilized complex-said analyte-said first immobilized reactant on said substrate without any material exchange between said controllably closed space and the outside environment.

Any suitable material can be used in the present devices. For example, the suitable material can be a self seal chamber, a self seal gel or a plastic chamber.

Any suitable substrate can be used in the present devices. For example, the substrate can comprise a material selected from the group consisting of a silicon, a plastic, a glass, a quartz glass, a ceramic, a rubber, a metal, a polymer, a hybridization membrane, and a combination thereof. The surface of the substrate can be modified to contain a chemically reactive group or a biomolecule. Exemplary chemically reactive groups include —CHO, —NH₂, —SH, —S—S—, an epoxy group and a Tosyl group. Exemplary biomolecules include biotin, streptavidin, avidin, his-tag, strept-tag, histidine and protein A. Preferably, the substrate is part of a chip, e.g., a DNA chip.

The present devices can be used to analyze any analytes. Exemplary analytes include a cell, a cellular organelle, a virus, a molecule and an aggregate or complex thereof.

Any suitable reactant can be used as the first immobilized reactant. For example, the first immobilized reactant can be a cell, a cellular organelle, a virus, a molecule and an aggregate or complex thereof. Preferably, the first immobilized reactant specifically binds to the analyte. Also preferably, the first immobilized reactant is an antibody to an analyte or a nucleic acid complementary to an analyte nucleic acid. The first immobilized reactant can be immobilized on the substrate via any suitable methods, e.g., via a chemically reactive group or a biomolecule contained on the surface of the substrate.

The labeled unimmobilized complex can comprise a detectable label on the second reactant or on the microparticle. The detectable label can be any suitable label such as a radioactive label, a fluorescent label, a chemical label, an enzymatic label, a luminescent label, a fluorescence resonance energy transfer (FRET) label and a molecular beacon. Preferably, the detectable label is a fluorescent label. Also preferably, the fluorescent label is adjacent to a second fluorescent label to generate the fluorescent signal. Exemplary fluorescent labels include FAM, TET, HEX, FITC, Cy3, Cy5, Texas Red, ROX, Fluroscein, TAMRA and a nanoparticle comprising a rare-earth metal. Alternatively, the microparticle in the unimmobilized complex functions as a directly detectable label that can be used to assess the presence and/or amount of the analyte.

Any suitable reactant can be used as the second reactant. Exemplary second reactants include a cell, a cellular organelle, a virus, a molecule and an aggregate or complex thereof. Preferably, the second reactant specifically binds to the analyte. Also preferably, the second reactant is an antibody to an analyte or a nucleic acid complementary to an analyte nucleic acid. The second reactant can be conjugated to the microparticle via any suitable methods, e.g., via a chemically reactive group or a biomolecule contained on the surface of the microparticle. Exemplary chemically reactive groups include —CHO, —NH₂, —SH, —S—S—, an epoxy group and a Tosyl group. Exemplary biomolecules include biotin, streptavidin, avidin, his-tag, strept-tag, histidine and protein A.

Any suitable microparticle can be used. Preferably, the microparticle is a magnetic, a magnetizable, an electrically charged or an electrically chargeable microparticle. The microparticle can comprise any suitable material such as an organic material, a glass, a SiO₂, a ceramic, a carbon and a metal. The microparticle can have any suitable size, e.g., having a diameter ranging from about 1 nm to about 20 μm.

In one specific embodiment, the microparticle used in the labeled unimmobilized complex is a magnetic microbead. The magnetic microbeads can be prepared by any suitable methods. For example, the methods disclosed in CN O/109870.8 or WO02/075309 can be used. Any suitable magnetizable substance can be used to prepare the magnetic microbeads useful in the present devices and methods. No-limiting examples of the magnetizable substances include ferrimagnetic substance, ferromagnetic substance, paramagnetic substance or superparamagnetic substances. In a specific embodiment, the magnetic microbeads comprise a paramagnetic substance, e.g., a paramagnetic metal oxide composition. Preferably, the paramagnetic metal oxide composition is a transition metal oxide or an alloy thereof. Any suitable transition metals can be used, such as iron, nickel, copper, cobalt, manganese, tantalum (Ta), zinc and zirconium (Zr). In a preferred embodiment, the metal oxide composition is Fe₃O₄ or Fe₂O₃. In another example, the magnetizable substance used in the magnetic microbeads comprises a metal composition. Preferably, the metal composition is a transition metal composition or an alloy thereof such as iron, nickel, copper, cobalt, manganese, tantalum, zirconium and cobalt-tantalum-zirconium (CoTaZr) alloy.

The magnetic microbeads may be prepared from the available primary beads, from raw materials or from metal oxides that are encapsulated by monomers which when crosslinked form rigid, polymeric coatings as disclosed in U.S. Pat. No. 5,834,121. As used herein, “rigid” refers to a polymeric coating that is cross linked to the extent that the polymeric coating stabilizes the metal oxide particle within the coating (i.e. the coating essentially does not swell or dissolve) so that the particle remains enclosed therein. As used herein, “microporous” refers to a resinous polymeric matrix that swells or expands in polar organic solvent. As used herein, “load” is used to mean the capacity of the bead for attachment sites useful for functionalization or derivatization.

Suitable substances which may be incorporated as magnetizable materials, for example, include iron oxides such as magnetite, ferrites of manganese, cobalt, and nickel, hematite and various alloys. Magnetite is the preferred metal oxide. Frequently, metal salts are taught to be converted to metal oxides then either coated with a polymer or adsorbed into a bead comprising a thermoplastic polymer resin having reducing groups thereon. When starting with metal oxide particles to obtain a hydrophobic primary bead, it is necessary to provide a rigid coating of a thermoplastic polymer derived from vinyl monomers, preferably a cross-linked polystyrene that is capable of binding or being bound by a microporous matrix. Magnetic particles may be formed by methods known in the art, e.g., procedures shown in Vandenberge et al., J. of Magnetism and Magnetic Materials, 15-18:1117-18 (1980); Matijevic, Acc. Chem. Res., 14:22-29 (1981); and U.S. Pat. Nos. 5,091,206; 4,774,265; 4,554,088; and 4,421,660. Examples of primary beads that may be used in this invention are shown in U.S. Pat. Nos. 5,395,688; 5,318,797; 5,283,079; 5,232,7892; 5,091,206; 4,965,007; 4,774,265; 4,654,267; 4,490,436; 4,336,173; and 4,421,660. Or, primary beads may be obtained commercially from available hydrophobic or hydrophilic beads that meet the starting requirements of size, sufficient stability of the polymeric coating to swell in solvents to retain the paramagnetic particle, and ability to adsorb or absorb the vinyl monomer used to form the enmeshing matrix network. Preferably, the primary bead is a hydrophobic, polystyrene encapsulated, paramagnetic bead. Such polystyrene paramagnetic beads are available from Dynal, Inc. (Lake Success, N.Y.), Rhone Poulonc (France), and SINTEF (Trondheim, Norway). The use of toner particles or of magnetic particles having a first coating of an unstable polymer which are further encapsulated to produce an exterior rigid polymeric coating is also contemplated.

The various means can move analytes and other items using any suitable force. In one example, the first means controllably moves the analyte to the first immobilized reactant via an electric, a magnetic, an acoustic, a gravitational or a centrifugational force. In another example, the second means controllably moves the analyte to the labeled unimmobilized complex via an electric, a magnetic, an acoustic, a gravitational or a centrifugational force. The second means controllably can move the analyte to the labeled unimmobilized complex by exerting a force on the microparticle of the labeled unimmobilized complex. In still another example, the third means controllably moves the labeled unimmobilized complex unbound to the analyte away from the first immobilized reactant via an electric, a magnetic, an acoustic, a gravitational or a centrifugational force. Preferably, the third means controllably moves the labeled unimmobilized complex unbound to the analyte away from the first immobilized reactant by exerting a force on the microparticle of the labeled unimmobilized complex.

In one specific embodiment, the analyte is a DNA, a RNA, a peptide nucleic acid (PNA), a locked nucleic acid (LNA), a protein, a peptide, an antibody and a polysaccharide. Preferably, the DNA, RNA, PNA and LNA has a length ranging from about 5 basepairs to about 1,000 basepairs. In another specific embodiment, the present devices are used to analyze DNA-DNA hybridization, DNA-RNA hybridization, DNA-LNA hybridization, DNA-PNA hybridization, RNA-RNA hybridization, RNA-PNA hybridization, RNA-LNA hybridization, PNA-PNA hybridization, PNA-LNA hybridization, protein-protein interaction, protein-nucleic-acid interaction, protein-polysaccharide interaction or antigen-antibody interaction.

The present devices can comprise a single or multiple analytic paths, e.g., from about 1 to about 10,000 analytic paths.

The present devices can further comprise a temperature control means. Exemplary temperature control means can comprise a PCR machine, an in situ PCR thermal cycler, a water bath or a micro thermal-controller.

The present devices can further comprise a means for detecting the sandwich of the labeled unimmobilized complex-the analyte-the first immobilized reactant. Exemplary detecting means can comprise a microscope, an optical scanner or fluorescent scanner.

### C. Methods for Analyzing an Analyte

In another aspect, the present invention is directed to a method for analyzing an analyte, which method comprises: a) providing an above-described device; b) introducing a sample containing or suspected of containing an analyte and a labeled unimmobilized complex comprising a second reactant capable of binding to said analyte and a microparticle into said controllably closed space of said device; c) operating said means of said device to form a sandwich of said labeled unimmobilized complex-said analyte-said first immobilized reactant on said substrate of said device without any material exchange between said controllably closed space and the outside environment; d) assessing said sandwich to determine presence and/or quantity of said analyte in said sample.

The present methods can be used to analyze any sample such as a solid, liquid or gas sample. The present methods can be used to analyze a single or multiple analytes, e.g., from about 1 to about 30,000 analytes. The multiple analytes can be analyzed sequentially or simultaneously.

In one specific embodiment, the sandwich of the labeled unimmobilized complex-the analyte-the first immobilized is formed by first moving the analyte to the labeled unimmobilized complex using the second means, allowing the analyte to bind to the labeled unimmobilized complex, and then moving the bound analyte-labeled unimmobilized complex to the first immobilized reactant using the first means, and allowing the bound analyte-labeled unimmobilized complex to bind to the first immobilized reactant to form the sandwich.

In another specific embodiment, the microparticle in the labeled unimmobilized complex itself functions as a directly detectable label.

The present methods can be used to assay any analyte, e.g., a cell, a cellular organelle, a virus, a molecule and an aggregate or complex thereof. Exemplary cells include animal cells, plant cells, fungus cells, bacterium cells, recombinant cells and cultured cells. Animal, plant, fungus, bacterium cells can be derived from any genus or subgenus of the Animalia, Plantae, fungus or bacterium kingdom. Cells derived from any genus or subgenus of ciliates, cellular slime molds, flagellates and microsporidia can also be assayed by the present methods. Cells derived from birds such as chickens, vertebrates such as fish and mammals such as mice, rats, rabbits, cats, dogs, pigs, cows, ox, sheep, goats, horses, monkeys and other non-human primates, and humans can be assayed by the present methods.

For animal cells, cells derived from a particular tissue or organ can be assayed by the present methods. For example, connective, epithelium, muscle or nerve tissue cells can be assayed. Similarly, cells derived from an accessory organ of the eye, annulospiral organ, auditory organ, Chievitz organ, circumventricular organ, Corti organ, critical organ, enamel organ, end organ, external female genital organ, external male genital organ, floating organ, flower-spray organ of Ruffini, genital organ, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmüller, sense organ, organ of smell, spiral organ, subcommissural organ, subfornical organ, supernumerary organ, tactile organ, target organ, organ of taste, organ of touch, urinary organ, vascular organ of lamina terminalis, vestibular organ, vestibulocochlear organ, vestigial organ, organ of vision, visual organ, vomeronasal organ, wandering organ, Weber organ and organ of Zuckerkandl can be used. Preferably, cells derived from an internal animal organ such as brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc can be assayed. Further, cells derived from any plants, fungi such as yeasts, bacteria such as eubacteria or archaebacteria can be assayed. Recombinant cells derived from any eucaryotic or prokaryotic sources such as animal, plant, fungus or bacterium cells can also be assayed. Body fluid such as blood, urine, saliva, bone marrow, sperm or other ascitic fluids, and subfractions thereof, e.g., serum or plasma, can also be assayed.

Exemplary cellular organelles include nuclei, mitochondria, chloroplasts, ribosomes, ERs, Golgi apparatuses, lysosomes, proteasomes, secretory vesicles, vacuoles and microsomes. Exemplary molecules include inorganic molecules, organic molecules and a complex thereof. Exemplary organic molecules include amino acids, peptides, proteins, nucleosides, nucleotides, oligonucleotides, nucleic acids, vitamins, monosaccharides, oligosaccharides, carbohydrates, lipids and a complex thereof.

Any amino acids can be assayed by the present methods. For example, a D- and a L-amino-acid can be assayed. In addition, any building blocks of naturally occurring peptides and proteins including Ala (A), Arg (R), Asn (N), Asp (D), Cys (C), Gln (Q), Glu (E), Gly (G), His (H), Ile (I), Leu (L), Lys (K), Met (M), Phe (F), Pro (P) Ser (S), Thr (T), Trp (W), Tyr (Y) and Val (V) can be assayed by the present methods.

Any proteins or peptides can be assayed by the present methods. For example, enzymes, transport proteins such as ion channels and pumps, nutrient or storage proteins, contractile or motile proteins such as actins and myosins, structural proteins, defense protein or regulatory proteins such as antibodies, hormones and growth factors can be assayed. Proteineous or peptidic antigens can also be assayed.

Any nucleic acids, including single-, double and triple-stranded nucleic acids, can be assayed by the present methods. Examples of such nucleic acids include DNA, such as A-, B- or Z-form DNA, and RNA such as mRNA, tRNA and rRNA.

Any nucleosides can be assayed by the present methods. Examples of such nucleosides include adenosine, guanosine, cytidine, thymidine and uridine. Any nucleotides can be assayed by the present methods. Examples of such nucleotides include AMP, GMP, CMP, UMP, ADP, GDP, CDP, UDP, ATP, GTP, CTP, UTP, dAMP, dGMP, CMP, dTMP, dADP, dGDP, dCDP, dTDP, dATP, dGTP, dCTP and dTTP.

Any vitamins can be assayed by the present methods. For example, water-soluble vitamins such as thiamine, riboflavin, nicotinic acid, pantothenic acid, pyridoxine, biotin, folate, vitamin B₁₂ and ascorbic acid can be assayed. Similarly, fat-soluble vitamins such as vitamin A, vitamin D, vitamin E, and vitamin K can be assayed.

Any monosaccharides, whether D- or L-monosaccharides and whether aldoses or ketoses, can be assayed by the present methods. Examples of monosaccharides include triose such as glyceraldehyde, tetroses such as erythrose and threose, pentoses such as ribose, arabinose, xylose, lyxose and ribulose, hexoses such as allose, altrose, glucose, mannose, gulose, idose, galactose, talose and fructose and heptose such as sedoheptulose.

Any lipids can be assayed by the present methods. Examples of lipids include triacylglycerols such as tristearin, tripalmitin and triolein, waxes, phosphoglycerides such as phosphatidylethanolamine, phosphatidylcholine, phosphatidylserine, phosphatidylinositol and cardiolipin, sphingolipids such as sphingomyelin, cerebrosides and gangliosides, sterols such as cholesterol and stigmasterol and sterol fatty acid esters. The fatty acids can be saturated fatty acids such as lauric acid, myristic acid, palmitic acid, stearic acid, arachidic acid and lignoceric acid, or can be unsaturated fatty acids such as palmitoleic acid, oleic acid, linoleic acid, linolenic acid and arachidonic acid.

The present method can be used to assay any sample. For example, the present method can be used to assay a mammalian sample. Exemplary mammals include bovines, goats, sheep, equines, rabbits, guinea pigs, murine, humans, felines, monkeys, dogs and porcines. The present method can also be used to assay a clinical sample. Exemplary clinical samples include serum, plasma, whole blood, sputum, cerebral spinal fluid, amniotic fluid, urine, gastrointestinal contents, hair, saliva, sweat, gum scrapings and tissue from biopsies. Preferably, the present method can be used to assay a human clinical sample.

### D. Exemplary Embodiments

We developed a microparticle based biochip device for analyzing an analyte to address the limitations of conventional passive biochips, active biochips, and lab-on-chip systems. In this device, the conventional separated steps, e.g., signal labeling, biochemical reaction, and reaction detection, are integrated by the microparticles of the invention. The microparticles of the invention provide signal labeling, increase of the rate of biochemical reaction, and reduction of background noise in the assay. The integrated steps are carried out in a controllably closed space enclosed by a suitable material (e.g., a reaction chamber). The analyzing process only requires the steps of introducing a sample to be analyzed into the device and assessing the reaction on the chip directly through the reaction chamber. Since there is no material exchange between the chamber and the outside environment during the reaction, manual manipulation steps in the conventional system are simplified and contamination is avoided. In the meantime, the device includes a means for applying a force to control the movement of the microparticles in the chamber to facilitate the reaction. These microparticles serve as carriers for detectable signal or signal labeling and amplification. The device of the invention can be used for analyzing one analyte or multiple analytes. The analyte can be a DNA molecule, a protein, a RNA molecule, an antibody, a peptide, a polysaccharide, a cell, a virus, or any combination of them, etc. The device comprises a substrate having reactant capable of binding to an analyte immobilized on a surface of the substrate. The device also comprises a reaction chamber enclosed by a suitable material. The suitable material is biocompatible and does not inhibit any interactions between an analyte and a reactant, including, but not limited to, interactions between a DNA and a DNA, a DNA and a RNA, a LNA and a DNA, a LNA and a RNA, a PNA and a DNA, a PNA and a RNA, a protein and a protein, an antibody and an antigen, a protein and a DNA, and a protein and a polysaccharide. The device can be combined with micro-device for sample preparation, portable temperature control system and detection system to form a portable analyte assay system. The device of the invention provides several advantages: automation, simplified manual manipulations, reduced possibility of contamination, enrichment of the analyte around the immobilized reactants for improved reaction efficiency and sensitivity. The microparticle of the invention can have a detectable label which can be detected by visible light under microscope or even detectable by a naked eye without the requirement of using expensive fluorescent scanner. Since the substrate is not integrated with the controlling means of the device, the substrate can be produced by conventional large-scale manufacture and thus the cost of manufacturing the substrates is low. Some embodiments of the device are described in more detail below and in the Examples.

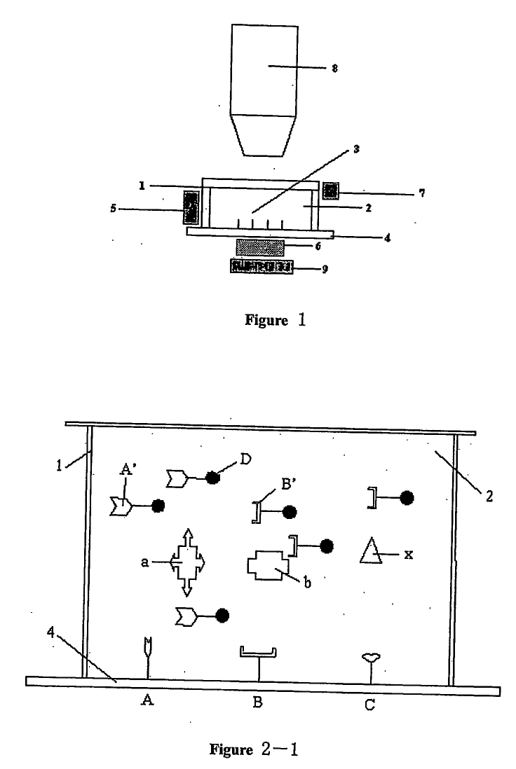

Some embodiments of the device of the invention are shown in FIG. 1. Referring to FIG. 1, reaction chamber 1 can be open and sealed. The suitable material is biocompatible and does not inhibit interaction between a DNA and a DNA, a DNA and a RNA, a LNA and a DNA, a LNA and a RNA, a PNA and a DNA, a PNA and a RNA, a protein and a protein, an antigen and an antibody, a protein and a nucleic acid, a protein and a polysaccharide. Some known materials include but not limited to a self seal chamber (MJ Research, Inc., MA, U.S.A.), a self seal gel (MJ Research, Inc., MA, U.S.A.), and a plastic sealed chamber. Reaction system 2 (FIG. 1) includes the microparticle having a reactant immobilized on the microparticle, the sample containing the analyte, and a reaction solution suitable for the interaction between the reactant and the analyte. Reactant 3 can be immobilized on the surface of substrate 4 by covalent reaction or non-covalent interaction between reactant 3 and the surface of the substrate modified to contain a chemically reactive group or a biomolecule. Solid substrate 4 should be a material that is thermoconductive, biocompatible, and easily obtainable. The material that can be used for the substrate includes but not limited to a glass, a quartz glass, a silicon, a ceramic, a metal, and a plastic. Device 5 is used for applying a force to the reaction chamber to promote movement of the microparticle in the chamber for enhancing interaction between the reactant immobilized on the microparticle and the analyte. Device 6 is used for applying a force to the reaction chamber to promote enrichment of the microparticle and the analyte bound to the microparticle around the reactant immobilized on the substrate in order to facilitate the interaction between the analyte and the reactant immobilized on the substrate. Device 7 is used for applying a force to the reaction chamber to move microparticle unbound to the reactant immobilized on the substrate away from the reactant to eliminate nonspecific signal on the substrate. Device 8, used for detection of the reaction, can be microscope for detecting visible light or a commercial fluorescent scanner for detecting a fluorescent signal. Device 9 is a reaction temperature control device, which can regulate rate of increasing temperature and sensitivity of temperature control. This temperature device can be a commercial PCR machine, an in situ PCR machine, a water bath equipment, or a micro-temperature control device for miniaturizing the entire device. The structure and the relative position of each part of the device in FIG. 1 is for illustrative purposes only.

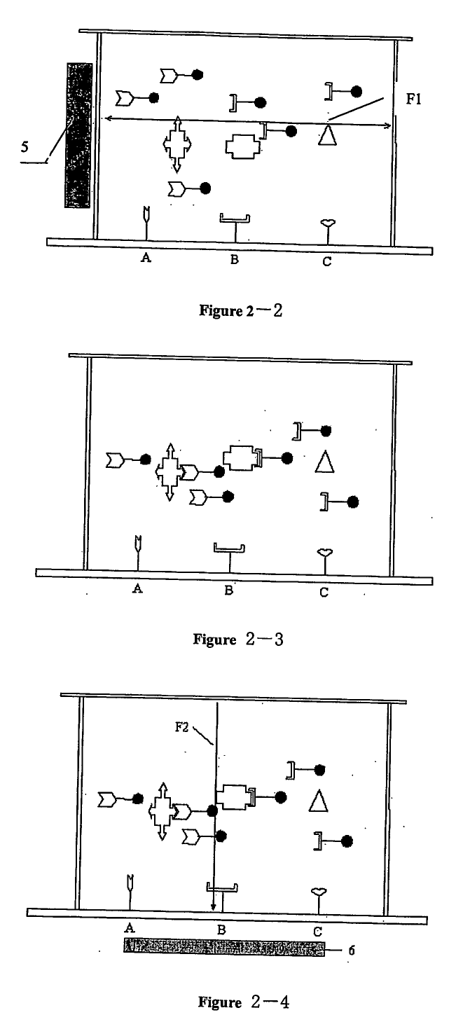

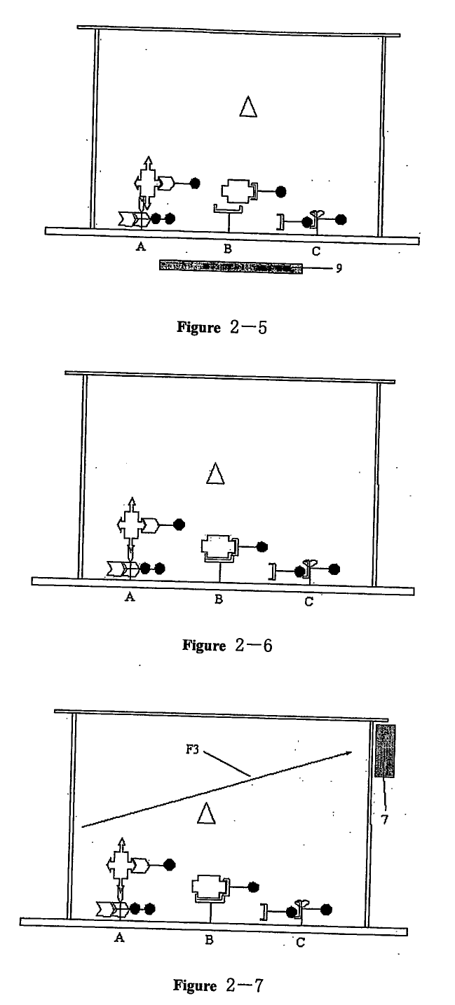

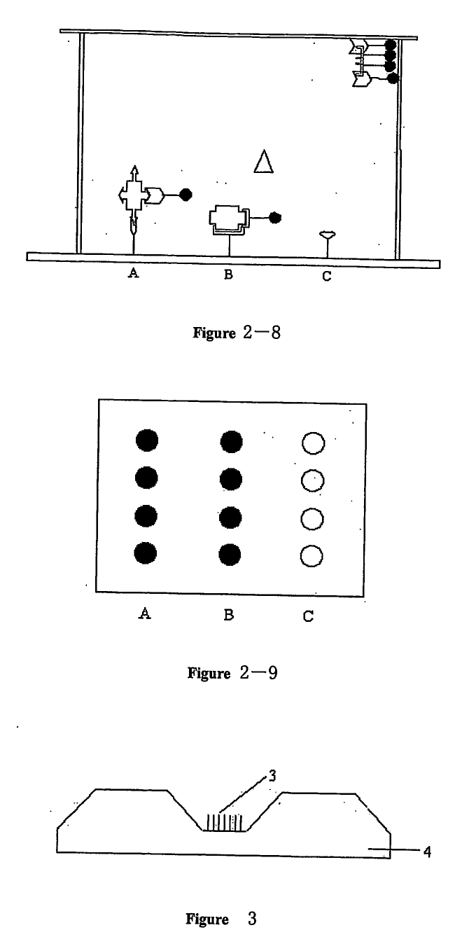

The following is an example of procedures for analyzing a sample using an exemplary device of the invention (FIG. 2):

(1) Referring to FIG. 2-1, a sample to be tested is mixed with a reaction solution to form reaction system 2, which is then introduced into the reaction chamber 1. Reaction system 2 includes a solution required for the reaction, microparticles having reactant A′ or B′ immobilized on the surface, and analyte a, b, and x in the sample. Substrate 4 has three different reactants, A, B, and C, immobilized at different locations on the surface. Reactant A′ and A can specifically bind to analyte a. Reactant B′ and B can specifically bind to analyte b.

(2) Referring to FIG. 2-2, device 5 is operated to generate a force F1 to accelerate movement of the microparticles in the reaction system 2. In the meantime, temperature control device 9 is used to control the temperature of the reaction system.

(3) Referring to FIG. 2-3, during the operation of device 5, analyte a or b binds respectively to reactant A′ or B′ immobilized on the microparticles. The operation of device 5 is then stopped.

(4) Referring to FIG. 2-4, device 6 is operated to generate a force F2 to drive the microparticles associated with the analytes towards the reactants immobilized on substrate 4.

(5) Referring to FIG. 2-5, during the operation of device 6, the microparticles associated with the analytes are enriched around the reactants immobilized on substrate 4. The operation of device 6 is stopped and device 9 is turned on to control the temperature in the reaction system.

(6) Referring to FIG. 2-6, analyte a or b associated with the microparticles binds respectively to reactant A or B immobilized on the substrate 4. The reaction is then stopped.

(7) Referring to FIG. 2-7, device 7 is operated to generate a force F3 to move microparticles unbound to substrate 4 away from the place near the reactants immobilized on substrate 4.

(8) Referring to FIG. 2-8, during the operation of device 7, the microparticles unbound to the substrate are removed from the place near the reactants immobilized on substrate 4 and are enriched in another area within reaction chamber 1. The operation of device 7 is stopped.

(9) Referring to FIG. 2-9, a detection device is used to assess the signals on substrate 4. The specific signal shown at the position of reactant A and reactant B, but not at the position of reactant C, on substrate 4 indicates that the sample tested contains analyte a and b.

Referring to the device shown in FIG. 1, in some embodiments, the microparticles are magnetic microparticles; device 5 is a magnetic, mechanical, or acoustic device for generating a magnetic, mechanical, or acoustic force; device 6 is a magnetic device for generating a magnetic force; device 7 is a magnetic or centrifugational device for generating a magnetic or a centrifugational force.

Referring to the device shown in FIG. 1, in other embodiments, the microparticles are polystyrene microparticles; device 5 is a mechanical or acoustic device for generating a mechanical or acoustic force; device 6 is a centrifugational device for generating a centrifugational force; device 7 is a centrifugational device for generating a centrifugational force. In these embodiments, when the side of the substrate having the reactants faces up, the area having the reactants sags relative to other part of the substrate as shown in FIG. 3, which facilitate enrichment of the microparticles around the reactants under centrifugational force.

Referring to the device shown in FIG. 1, in other embodiments, the microparticles re electrically charged microparticles; device 5 is an electric, mechanical, or acoustic device for generating an electric, mechanical, or acoustic force; device 6 is an electronic device for generating an electronic force; device 7 is an electronic or centrifugational device for generating an electronic or a centrifugational force.

### E. EXAMPLES

### Example 1 Detection of Nucleic Acid of Hepatitis B

1. Preparation of a Substrate Having Aldehyde Group

A glass substrate was soaked in an acidic wash solution at room temperature overnight. The glass substrate was then rinsed with water, washed three times with distilled water, and washed two times with deionized water. It was then dried by centrifugation followed by heating to 110° C. for 15 minutes. The glass substrate was soaked in 1% APTES in 95% ethanol and was shaken gently in a shaker for one hour at room temperature. After soaking in 95% ethanol, the glass substrate was rinsed and then dried in vacuum drier at −0.08 Mpa to −0.1 Mpa and 110° C. for twenty minutes. Once the glass substrate was cooled to room temperature, it was soaked in 12.5% glutaraldehyde solution (for 400 ml 12.5% glutaraldehyde solution, mix 100 ml 50% glutaraldehyde with 300 ml sodium phosphate buffer (1M NaH₂PO₄ 30 ml and 2.628 g NaCl, adjust pH to 7.0)). After soaking for 4 hours at room temperature, the solution was shaken gently and the glass substrate was taken out of the glutaraldehyde solution and washed once in 3×SSC, followed by twice in deionized water. The excess water was removed by centrifugation and the glass plate was dried at room temperature.

2. Synthesis of Primers and Reactants

The primers and the reactants are synthesized by Shanghai Boya Biotechnologies Shanghai BioAsia Biotechnology co. Reactant 1 is amino-5′-polyT(15nt) GCATGGACATCGACCCTTATAAAG-3′ (SEQ ID NO:1). Reactant 2 is Hex-5′-GGAGCTACTGTGGAGTTACTC CTGG-3′-Biotin (SEQ ID NO:2). The upstream primer is gTTCAAgCCTCCAAgCTgTg (SEQ ID NO:3). The down stream primer is TCAgAAggCAAAAAAgAgAgTAACT (SEQ ID NO:4).

3. Immobilization of Biotin Labeled Reactant on Magnetic Microparticles

Dynabeads® M-280 Streptavidin (10 mg/ml, Dynal Biotech ASA, Oslo, Norway) 100 [symbol] l (magnetic microparticles) were washed three times in 1×PBS (0.1% BSA). The washed microparticle were then resuspended in 100 [symbol] l 1×PBS (0.1% BSA) and mixed with 2 [symbol] l of reactant 2. The reaction was carried out for 30 minutes at 30° C. with continuous shaking so that the microparticles were not pelleted at the bottom. The microparticles were then washed three times in 1×TE buffer and were resuspended in 1×TE buffer.

4. Preparation of the Glass Substrate Having Reactant Immobilized on the Surface

Reactant 1 is dissolved in 50% DMSO with final concentration at 10 [symbol] M. The reactants were printed on the substrate using microarray printing device (Cartesian Technologies, CA, U.S.A.) according to a pre-designed pattern. The printed substrate was then dried overnight at room temperature. The printed substrate was then soaked twice in 0.2% SDS at room temperature for 2 minutes with shaking. The substrate was rinsed twice and washed once with deionized water and then dried by centrifugation. The substrate was then transferred to a NaBH₄ solution (0.1 g NaBH₄ dissolved in 300 ml 1×PBS and 100 ml ethanol) and shaken gently at room temperature for 5 minutes. The substrate was again rinsed twice and washed twice with deionized water with 1 minute for each wash and dried by centrifugation.

5. Preparation of Reaction Chamber

The reaction chamber was prepared using self seal chamber (MJ Research, Inc., MA, U.S.A.) according to the operation manual. The substrate having the printed reactants, e.g., the immobilized probes, were facing the inside of the chamber.

6. Nucleic Acid Template

The plasmid (pCP10, 100 ng/ [symbol] l) containing the sequence amplified using the upstream primer and the downstream primer was used as template.

7. Nucleic Acid Amplification

PCR reaction system included: 10 mmol/L Tris-HCl (pH 8.3 at 24° C.), 50 mmol/L KCl, 1.5 mmol/L MgCl₂, 0.5 [symbol] mol/L of upstream primer and downstream primer, 1 unit Taq DNA polymerase, 200 [symbol] mol/L dNTPs (DATP, dTTP, dCTP, and dGTP), 0.1% BSA, 0.1% Tween 20, 2 [symbol] mol/L reactant 2, 2 [symbol] l template. The total reaction volume is 25 [symbol] l. The PCR reaction system was then introduce into the reaction chamber and sealed. The PCR was carried using PTC-200 (MJ Research Inc.) with a program: predenaturing at 94° C. for 1 minute; main cycle at 94° C. for 30 sec, 55° C. for 30 sec, and 72° C. for 1 minute for 30 cycles; and at 72° C. for 10 minutes.

8. Hybridization

Microparticles (25 [symbol] l) prepared in section 3 of this example was centrifuged to remove the supernatant and was introduced into 25 [symbol] l of the PCR product in the reaction chamber. The reaction chamber was then sealed. The PCR product was first denatured for 2 minutes at 94° C., and then cooled immediately to 52° C. An altering magnetic field was applied to the reaction chamber to accelerate movement of the magnetic microparticles for 10 minutes. Then a magnetic field was applied right underneath the substrate to allow enrichment of the microparticles around the reactants. The chamber was incubated for 30 minutes to allow hybridization. A magnetic field was then applied to the chamber to move the unbound microparticles away from the immobilized reactants.

9. Detection

The light signal was detected using Leica transmission microscope according to the operation manual. A black dot signal at the position of the reactants immobilized on the substrate, but no signal at the negative control position and no signal on the substrate without adding the sample, indicated that the sample contains nucleic acid of hepatitis B. The fluorescent signal was detected by scanning with ScanArray 4000 (GSI Lumonics, MA, U.S.A.). The detection was carried out according to the operation manual with the following setting: laser wavelength at 543 nm, laser device 3, filter 7 for signal detection, 80% function of laser device and photoelectric multiplier, scanning focus adjusted based on the substrate. A strong fluorescent signal at the position of the reactants immobilized on the substrate, but no signal at the negative control position and no signal on the substrate without adding the sample, indicated that the sample contains nucleic acid of hepatitis B.

### Example 2 Detection of Nucleic Acid of Hepatitis C

The detection of nucleic acid of hepatitis C was carried via procedures describe similar to those described in Example 1 with the following modifications. Reactant 1 used for this example was amino-5′-polyT(15 nt) ACGACACTCATACTAACGCCA-3′ (SEQ ID NO:5). Reactant 2 was Hex-5′-GTCGTCCTGGCAATTCCG-3′-NH2 (SEQ ID NO:6). Upstream primer was 5′-CTCgCAAgCACCCTATCAggCAgT-3′ (SEQ ID NO:7). Downstream primer was 5′-gCAgAAAgCgTCTAgCCATggCgT-3′ (SEQ ID NO:8). Amino group modified reactant 2 was immobilized on magnetic microparticles (Dynabeads® M-270 Carboxylic Acid at 10 mg/ml, Dynal Biotech ASA, Oslo, Norway) according to the manufacture's manual. The nucleic acid template was isolated from fresh whole blood from samples that are hepatitis C positive shown by serological method using Roche High Pure™ Viral Nucleic Acid Kit according to product manual and then dissolved in 25 [symbol] l elution buffer. After the hybridization in the reaction chamber, the signal was detected using Leica transmission microscope. A black dot signal at the position of the reactants immobilized on the substrate, but no signal at the negative control position and no signal on the substrate without adding the sample, indicated that the sample contains nucleic acid of hepatitis C. The fluorescent signal was detected by scanning with ScanArray 4000 (GSI Lumonics, MA, U.S.A.). A strong fluorescent signal at the position of the reactants immobilized on the substrate, but no signal at the negative control position and no signal on the substrate without adding the sample, indicated that the sample contains nucleic acid of hepatitis C.

### Example 3 Detection of 16S rRNA of E coli

The detection of nucleic acid of 6S rRNA of E. coli was carried our similar to procedures describe in Example 1 with the following modifications. Reactant 1 used for this example was amino-5′-polyT(15 nt) GCAAA GGTAT TTACT TTACT CCC-3′ (SEQ ID NO:9). Reactant 2 was Hex-5′-AATCA CAAAG TCGTA AGCGC C-3′-Biotin (SEQ ID NO:10). The nucleic acid 16S rRNA was isolated from 100 [symbol] l E. coli DN5 (10,000/ml) cultured in LB medium using RNeasy Kit from QIAGEN (QIAGEN GmbH Germany) and was dissolved in 30 [symbol] l of a solution containing 5×SSC and 0.1% SDS. After hybridization, the signal was detected using Leica transmission microscope. A black dot signal at the position of the reactants immobilized on the substrate, but no signal at the negative control position and no signal on the substrate without adding the sample, indicated that the sample contains E. coli 16S rRNA. The fluorescent signal was detected by scanning with ScanArray 4000 (GSI Lumonics, MA, U.S.A.). A strong fluorescent signal at the position of the reactants immobilized on the substrate, but no signal at the negative control position and no signal on the substrate without adding the sample, indicated that the sample contains E. coli 16S rRNA.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A device for analyzing an analyte, which device comprises:
a) a controllably closed space enclosed by a suitable material on a substrate, wherein said suitable material is thermoconductive, biocompatible and does not inhibit binding between an analyte and a reactant, and said controllably closed space comprising, on the surface of said substrate, a first immobilized reactant capable of binding to said analyte;
b) a first means for controllably moving said analyte to said first immobilized reactant;
c) a second means for controllably moving said analyte to a labeled unimmobilized complex comprising a second reactant capable of binding to said analyte and a microparticle; and
d) a third means for controllably moving said labeled unimmobilized complex unbound to said analyte away from said first immobilized reactant,
and wherein addition of a sample comprising said analyte and said labeled unimmobilized complex into said controllably closed space and operation of said means result in formation of a sandwich of said labeled unimmobilized complex-said analyte-said first immobilized reactant on said substrate.

2. The device of claim 1, wherein the suitable material is a self seal chamber, a self seal gel or a plastic chamber.

3. The device of claim 1, wherein the substrate comprises a material selected from the group consisting of a silicon, a plastic, a glass, a quartz glass, a ceramic, a rubber, a metal, a polymer, a hybridization membrane, and a combination thereof.

4. The device of claim 1, wherein the surface of the substrate is modified to contain a chemically reactive group or a biomolecule.

5. The device of claim 4, wherein the chemically reactive group is selected from the group consisting of —CHO, —NH₂, —SH, —S—S—, an epoxy group and a Tosyl group.

6. The device of claim 4, wherein the biomolecule is selected from the group consisting of biotin, streptavidin, avidin, his-tag, strept-tag, histidine tag and protein A.

7. The device of claim 1, wherein the analyte is selected from the group consisting of a cell, a cellular organelle, a virus, a molecule and an aggregate or complex thereof.

8. The device of claim 1, wherein the first immobilized reactant is selected from the group consisting of a cell, a cellular organelle, a virus, a molecule and an aggregate or complex thereof.

9. The device of claim 1, wherein the first immobilized reactant specifically binds to the analyte.

10. The device of claim 1, wherein the first immobilized reactant is immobilized on the substrate via a chemically reactive group or a biomolecule contained on the surface of the substrate.

11. The device of claim 1, wherein the labeled unimmobilized complex comprises a detectable label on the second reactant or on the microparticle.

12. The device of claim 11, wherein the detectable label is selected from the group consisting of a radioactive label, a fluorescent label, a chemical label, an enzymatic label, a luminescent label, a fluorescence resonance energy transfer (FRET) label and a molecular beacon.

13. The device of claim 11, wherein the detectable label is a fluorescent label.

14. The device of claim 13, wherein the fluorescent label is adjacent to a second fluorescent label to generate the fluorescent signal.

15. The device of claim 13, wherein the fluorescent label is selected from the group consisting of FAM, TET, HEX, FITC, Cy3, Cy5, Texas Red, ROX, Fluroscein, TAMRA and a nanoparticle comprising a rare-earth metal.

16. The device of claim 1, wherein the second reactant is selected from the group consisting of a cell, a cellular organelle, a virus, a molecule and an aggregate or complex thereof.

17. The device of claim 1, wherein the second reactant specifically binds to the analyte.

18. The device of claim 1, wherein the second reactant is conjugated to the microparticle via a chemically reactive group or a biomolecule contained on the surface of the microparticle.

19. The device of claim 18, wherein the chemically reactive group is selected from the group consisting of —CHO, —NH₂, —SH, —S—S—, an epoxy group and a Tosyl group.

20. The device of claim 18, wherein the biomolecule is selected from the group consisting of biotin, streptavidin, avidin, his-tag, strept-tag, histidine tag and protein A.

21. The device of claim 1, wherein the microparticle is a magnetic, a magnetizable, an electrically charged or an electrically chargeable microparticle.

22. The device of claim 1, wherein the microparticle comprises a material selected from the group consisting of an organic material, a glass, a SiO₂, a ceramic, a carbon and a metal.

23. The device of claim 1, wherein the microparticle has a diameter ranging from about 1 nm to about 20 μm.

24. The device of claim 1, wherein the first means controllably moves the analyte to the first immobilized reactant via a force selected from the group consisting of electric, magnetic, acoustic, gravitational and centrifugational force.

25. The device of claim 1, wherein the second means controllably moves the analyte to the labeled unimmobilized complex via a force selected from the group consisting of electric, magnetic, acoustic, gravitational and centrifugational force.

26. The device of claim 25, wherein the second means controllably moves the analyte to the labeled unimmobilized complex by exerting a force on the microparticle of the labeled unimmobilized complex.

27. The device of claim 1, wherein the third means controllably moves the labeled unimmobilized complex unbound to the analyte away from the first immobilized reactant via a force selected from the group consisting of electric, magnetic, acoustic, gravitational and centrifugational force.

28. The device of claim 27, wherein the third means controllably moves the labeled unimmobilized complex unbound to the analyte away from the first immobilized reactant by exerting a force on the microparticle of the labeled unimmobilized complex.

29. The device of claim 1, wherein the analyte is selected from the group consisting of a DNA, a RNA, a peptide nucleic acid (PNA), a locked nucleic acid (LNA), a protein, a peptide, an antibody and a polysaccharide.

30. The device of claim 29, wherein the DNA, RNA, PNA and LNA has a length ranging from about 5 basepairs to about 1,000 basepairs.

31. The device of claim 1, which is used to analyze DNA-DNA hybridization, DNA-RNA hybridization, DNA-LNA hybridization, DNA-PNA hybridization, RNA-RNA hybridization, RNA-PNA hybridization, RNA-LNA hybridization, PNA-PNA hybridization, PNA-LNA hybridization, protein-protein interaction, protein-nucleic-acid interaction, protein-polysaccharide interaction or antigen-antibody interaction.

32. The device of claim 1, which comprises a single or multiple analytic paths.

33. The device of claim 1, which comprises from about 1 to about 10,000 analytic paths.

34. The device of claim 1, which further comprises a temperature control means.

35. The device of claim 34, wherein the temperature control means comprises a PCR machine, an in situ PCR thermal cycler, a water bath or a micro thermal-controller.

36. The device of claim 1, which further comprises a means for detecting the sandwich of the labeled unimmobilized complex-the analyte-the first immobilized reactant.

37. The device of claim 36, wherein the detecting means comprises a microscope, an optical scanner or fluorescent scanner.

38. The device of claim 1, wherein the sandwich of the labeled unimmobilized complex-the analyte-the first immobilized reactant on the substrate without any material exchange between said controllably closed space and the outside environment.

39. A method for analyzing an analyte, which method comprises:
a) providing a device of claim 1;
b) introducing a sample containing or suspected of containing an analyte and a labeled unimmobilized complex comprising a second reactant capable of binding to said analyte and a microparticle into said controllably closed space of said device;
c) operating said means of said device to form a sandwich of said labeled unimmobilized complex-said analyte-said first immobilized reactant on said substrate;
d) assessing said sandwich to determine presence and/or quantity of said analyte in said sample.

40. The method of claim 39, wherein the sample is a solid, liquid or gas sample.

41. The method of claim 39, which is used to analyze a single or multiple analytes.

42. The method of claim 41, wherein the multiple analytes are analyzed sequentially or simultaneously.

43. The method of claim 39, which is used to analyze from about 1 to about 30,000 analytes.

44. The method of claim 39, wherein the sandwich of the labeled unimmobilized complex-the analyte-the first immobilized reactant is formed by first moving the analyte to the labeled unimmobilized complex using the second means, allowing the analyte to bind to the labeled unimmobilized complex, and then moving the bound analyte-labeled unimmobilized complex to the first immobilized reactant using the first means, allowing the bound analyte-labeled unimmobilized complex to bind to the first immobilized reactant to form the sandwich, and controllably moving the labeled unimmobilized complex unbound to the analyte away from the first immobilized reactant using the third means.

45. The method of claim 39, wherein the microparticle in the labeled unimmobilized complex itself functions as a directly detectable label.

46. The method of claim 39, wherein the sandwich of the labeled unimmobilized complex-the analyte-the first immobilized reactant on the substrate without any material exchange between said controllably closed space and the outside environment.
